(19) United States
(12) Patent Application Publication
(10) Pub. No.: US 2006/0004199 A1
(43) Pub. Date: Jan. 5, 2006

(54) Process for the preparation of pure 3-[2-[4-(6-fluoro-1, 2-benzisoxazol-3-yl)-1-piperidinyl]-ethyl]-6,7,8,9-tetrahydro-2-methyl-4H-pyrido [1,2-a] pyrimidin-4-one

(75) Inventors: Buchi Reddy Reddy, Hyderabad (IN); Sunkari Sudhakar, Hyderabad (IN); Ramesh Chakka, Sccundarabad (IN); Tamma Ranga Reddy, Hyderabad (IN); Kandirelli Venkata Kiran Kumar, Guntur (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/883,579

(22) Filed: Jul. 1, 2004

(51) Int. Cl. C07D 487/04

(52) U.S. Cl. 544/281

(57) ABSTRACT

A process for the preparation of 3-[2-[4-(6-fluoro-1,2-benzisoxazol-3-yl)-1-piperidinyl]-ethyl]-6,7,8,9-tetrahydro-2-methyl-4H-pyrido[1,2-a]pyrimidin-4-one, which is known as Risperidone of Formula (I). Risperidone of Formula (I) is represented by the following structure.

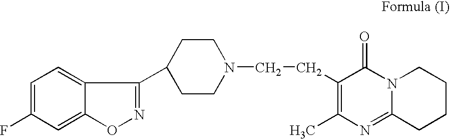

### FIELD OF THE INVENTION

The present invention relates to an improved process for the preparation of pure 3-[2-[4-(6-fluoro-1,2-benzisoxazol-3-yl)-1-piperidinyl]-ethyl]-6,7,8,9- tetrahydro-2-methyl-4H-pyrido[1,2-a]pyrimidin-4-one, which is known as Risperidone of Formula (I). It is marketed under brand name “risperidal”. Risperidone of Formula (I) of the present invention is represented by the following structure.

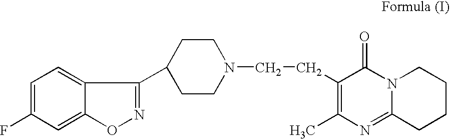

Risperidone is useful as antipsychotic agent in the treatment of psychotic disorders.

### BACKGROUND OF THE INVENTION

EP 196132 B1 describes certain 1,2-benzisoxazol-3-yl derivatives having psychotic and anti-serotonin activity including 3-[2-[4-(6-Fluoro-1,2-benzisoxazol-3-yl)-1-piperidinyl]-ethyl]-6,7,8,9-tetrahydro-2-methyl-4H-pyrido[1,2-a]pyrimidin-4-one (Risperidone), which is a mixed 5-HT₂A/D2-receptor antagonist and is an example of typical neuroleptic drug.

EP 196132 B1 exemplified the process for the preparation of Risperidone, which includes the condensation between 3-(2-chloroethyl)-6,7,8,9-tetrahydro-2-methyl-4H-pyrido[1,2-a]pyrimidin-4-one mono hydrochloride and 6-fluoro-3-(4-piperidinyl)-1,2-benzisoxazole in the presence of N,N-dimethyl formamide, sodium carbonate and catalytic amount of potassium iodide. The crude risperidone product was crystallized from a mixture of DMF and isopropanol to yield of 46% of Risperidone.

U.S. 2002/0115672 and U.S. 2002/0115673 describes the process for the preparation of Risperidone, which also includes the processes for the preparation of crystalline Form-A, Form-B and Form-E. The process for the preparation of Risperidone comprises the condensation of 6-fluoro-3-(4-piperidinyl)-1,2-benzisoxazole of the Formula (II) 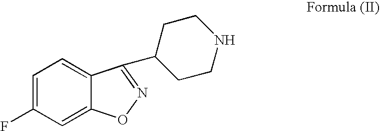
and 3-(2-chloroethyl)-6,7,8,9-tetrahydro-2-methyl-4H-pyrido[1,2-a]pyrimidin-4-one of the Formula (III) 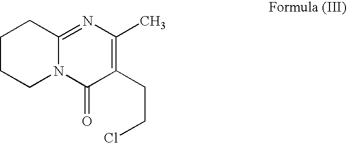
in acetonitrile, further, the resulting crude mass was recrystallised from alcohols to get Risperidone.

EP 196132 B1 and U.S. 2002/0115672 were suffering with many disadvantages like involving multiple operation, recovery stages and poor quality. Moreover, the product obtained from described process suffering with non-controllable impurity like “3-[2-[4-(6-Fluoro-1,2-benzisoxazol-3-yl)-1-piperidinylcarbonyloxy]ethyl]-6,7,8,9-tetrahydro-2-methyl-4H-pyrido[1,2-a]pyrimidin-4-one”, which is commonly referred as carboxylate impurity.

Hence, the object of the present invention is to provide an improved process for the preparation of substantially pure Risperidone, which is simple, cost-effective, eco-friendly and commercially suitable process by overcoming the problems encountered in the above prior art process.

The free flowing solids are in general preferred for pharmaceutical applications. Apparently, the present inventive substance was pure crystalline in nature, free from residual solvents and it can be readily used for pharmaceutical applications without proceeding for further purification.

### SUMMERY OF THE INVENTION

The present invention comprises, condensation of 3-(2-Chloroethyl)-6,7,8,9-tetrahydro-2 methyl-4H-pyrido[1,2-a]pyrimidin-4-one and 6-Fluoro-3-(4-piperidinyl)-1,2-benzisoxazole in presence a tertiary amine base like N ethyl diisopropyl amine in lower alcohols like methanol.

Another objective of the present invention is to minimize the level of impurities like “3-[2-[4-(6-fluoro-1,2-benzisoxazol-3-yl)-1-piperidinyl]ethyl]-9-hydroxy-6,7,8,9-tetrahydro-2-methyl-4H-pyrido[1,2-a]pyrimidin-4-one and “3-[2-[4-(6-fluoro-1,2-benzisoxazol-3-yl)-1-piperidinylcarbonyloxy]ethyl]-6,7,8,9-tetrahydro-2-methyl-4H-pyrido[1,2-a]pyrimidin-4-one”) in desired final product by recrystallization in aqueous alcohols

Thus the process of the present invention is simple, reproducible, cost-effective, eco-friendly non-hazardous and hence can be well suited for large-scale production.

### DESCRIPTION OF FIGURES

FIG. 1 is an X-ray powder diffractogram of a Risperidone crystalline form prepared by according to the present invention.

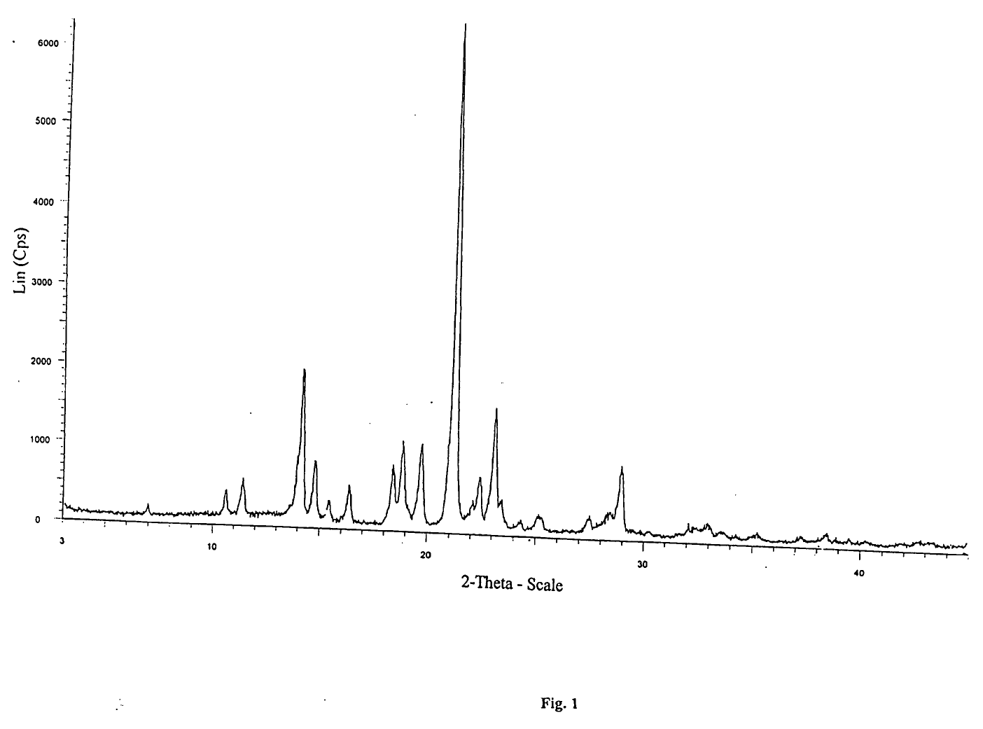

FIG. 2 is a differential scanning calorimetric thermogram of a Risperidone crystalline form prepared by according to the present invention.

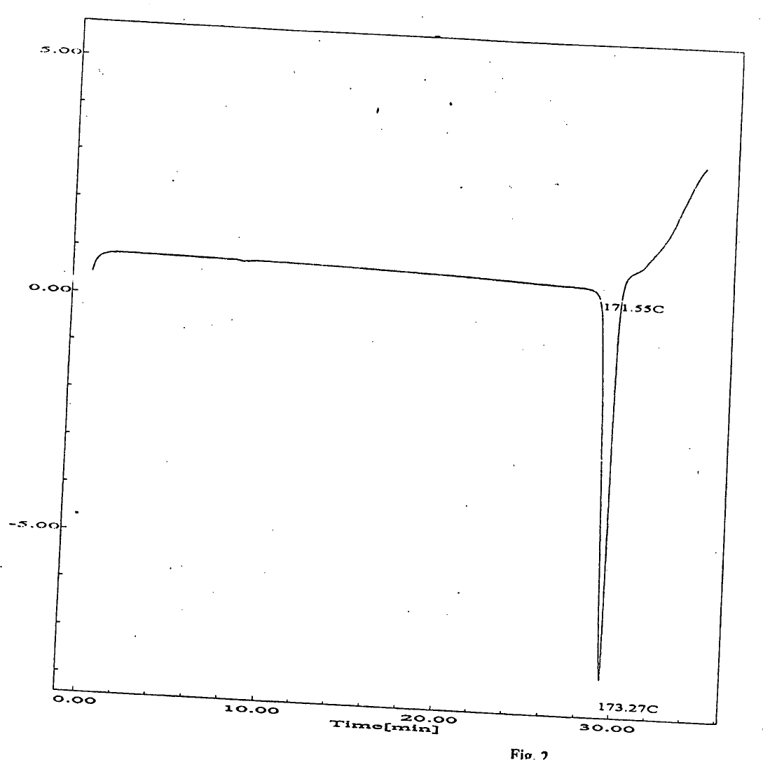

FIG. 3 is an infrared absorption spectrum of a Risperidone crystalline form prepared by according to the present invention

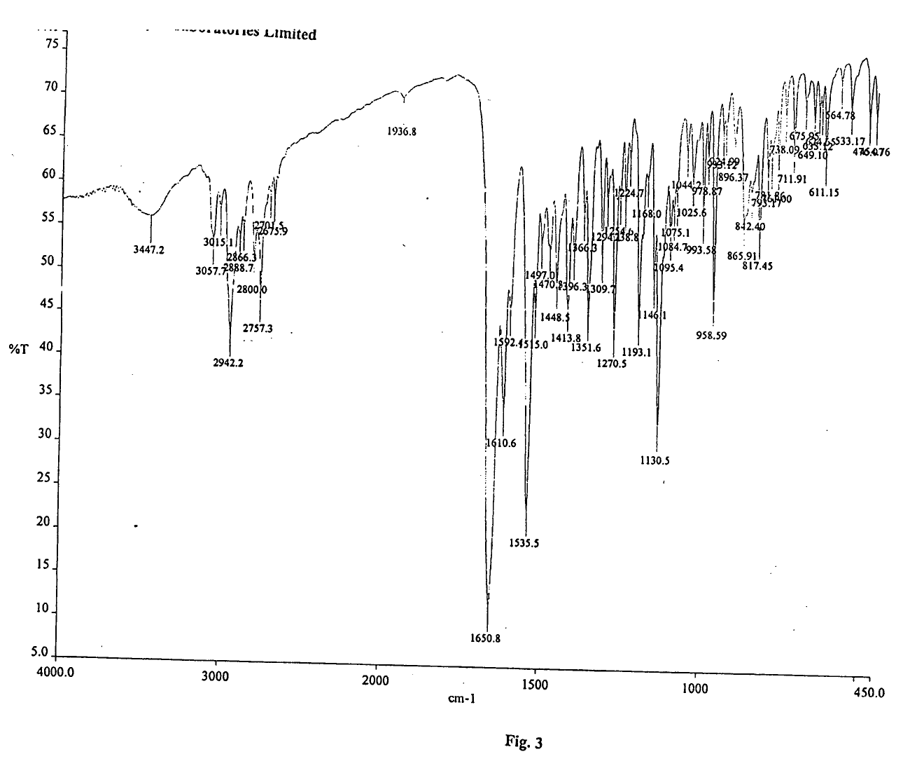

### DESCRIPTION OF THE INVENTION

The present invention related to process for the preparation of 3-[2-[4-(6-Fluoro-1,2-benzisoxazol-3-yl)-1-piperidinyl]-ethyl]-6,7,8,9-tetrahydro-2-methyl-4H-pyrido [1,2-a]pyrimidin-4-one which comprises;
a) dissolving 3-(2-Chloroethyl)-6,7,8,9-tetrahydro-2-methyl-4H-pyrido [1,2-a]pyrimidin-4-one and 6-Fluoro-3-(4-piperidinyl)-1,2-benzisoxazole in lower alcohols like methanol, ethanol, propanol;
b) adding an organic base like N ethyl diisopropyl amine to the above step (a) solution;
c) heating the resulting solution of step (b) upto 45 to 50° C.;
d) maintaining the reaction mass of step (c) for about 55 to 80 hours;
e) cooling the above solution of step (d) upto 25 to 35° C.;
f) separated solids from step (e) were filtered and washed with methanol;
g) slurring the wet filtered solids of step (f) with water for about 30 to 60 minutes at 25 to 35° C.;
h) filtering the solids of step (g), followed by washing with water and then spin dried for 30 to 60 minutes;
i) wet compound further dried at 70 to 75° C. for 4 hours under reduced pressure;
j) loading the resulting compound in 5-15% aqueous ethanol;
k) heating the resulted mass upto 40 to 45° C. for complete dissolution;
l) maintaining the above solution for about 20 minutes at the same temperature and filtered out under nitrogen atmosphere;
m) cooling the solution to 0 to 5° C. and then maintained for about 30 to 60 minutes;
n) centrifuging the solid of step of (m);
o) washing the separated solids of step (n) with chilled 5% aqueous ethanol and spin dried for 30 to 60 minutes to get 3-[2-[4-(6-Fluoro-1,2 -benzisoxazol-3-yl)-1-piperidinyl]-ethyl]-6,7,8,9-tetrahydro-2-methyl-4H-pyrido [1,2-a]pyrimidin-4-one.

Crystalline form of the Risperidone of the present invention is free flowing solid.

The present invention is illustrated by the following examples, which are not intended to limit the effective scope of the invention.

### Process for the Preparation of 3-[2-[4-(6-FLUORO-1,2-benzisoxazol-3-yl)-1-piperidinyl]-ethyl]-6,7,8,9-tetrahydro-2-methyl-4H-pyrido[1,2-A]pyrimidin-4-one

### EXAMPLE 1

6-Fluoro-3-(4-piperidinyl)-1,2-benzisoxazole (20.0 grams), 3-(2-chloroethyl)-6,7,8,9-tetrahydro-2-methyl-4H-pyrido[1,2-a]pyrimidin-4-one (22.6 grams) and diisopropyl ethylamine (14.1 grams) were dissolved in methanol (90.0 ml). The resulting reaction mixture was maintained at 45-50° C. for about 70-100 hours. The reaction mass was then cooled to 25-35° C. and then separate solid product. The solid mass was filtered and washed with methanol (20.0 ml) followed by water (120.0 ml). The wet product was dried at 70-80° C. to get 3-[2-[4-(6-fluoro-1,2-benzisoxazol-3-yl)-1-piperidinyl]-ethyl]-6,7,8,9-tetrahydro-2-methyl-4H-pyrido[1,2-A]pyrimidin-4-one. (Yield: 29.0 g; 77.8%; purity by HPLC is 99.93%)

### EXAMPLE 2

6-Fluoro-3-(4-piperidinyl)-1,2-benzisoxazole (10 Kg), 3-(2-chloroethyl)-6,7,8,9-tetrahydro-2-methyl-4H-pyrido[1,2-a]pyrimidin-4-one (11.3 Kg) and diisopropyl ethylamine (7 Kg) were dissolved in methanol (45 Lt). The resulting reaction mixture was maintained at 45-50° C. for about 70 to 100 hours. The reaction mass was then cooled to 25-35° C. and then separate solid product. The solid mass was filtered and washed with methanol (10 Lt) followed by water (60 Lt). The wet product was dried at 70-80° C. to get 3-[2-[4-(6-fluoro-1,2-benzisoxazol-3-yl)-1-piperidinyl]-ethyl]-6,7,8,9-tetrahydro-2-methyl-4H-pyrido[1,2-A]pyrimidin-4-one. (Yield: 18 Kg, purity by HPLC is 99.93%).

### Purification of 3-[2-[4-(6-fluoro-1,2-benzisoxazol-3-yl)-1-piperidinyl]-ethyl]-6,7,8,9-tetrahydro-2-methyl-4H-pyrido [1,2-A]pyrimidin-4-one

### EXAMPLE 3

3-[2-[4-(6-Fluoro-1,2-benzisoxazol-3-yl)-1-piperidinyl]-ethyl]-6,7,8,9-tetrahydro-2-methyl-4H-pyrido[1,2-a]pyrimidin-4-one (15 Kg) was charged into 5% aqueous ethanol (180 Lt). The resulting mixture was heated up to 40 to 45° C. and maintained at the temperature upto 20 minutes. The solution was filtered through a cartridge filter at the same temperature. The clear solution was cooled to 0 to 5° C. and maintained for 30 to 60 minutes. The resulting crystalline material was centrifuged and washed with chilled 5% aqueous ethanol (7.5 Lt). The wet material was dried in a vacuum cone drier at 70 to 75° C. for about 7 hours. The hot material was cooled to room temperature.

We claim:

1. An improved process for the preparation of 3-[2-[4-(6-Fluoro-1,2-benzisoxazol-3-yl)-1-piperidinyl]ethyl]-6,7,8,9-tetrahydro-2-methyl-4H-pyrido[1,2-a]pyrimidin-4-one which comprises;
a) dissolving 3-(2-chloroethyl)-6,7,8,9-tetrahydro-2-methyl-4H-pyrido[1,2-a]pyrimidin-4-one and 6-fluoro-3-(4-piperidinyl)-1,2-benzisoxazole in lower alcohols;
b) adding an organic base like N-ethyl diisopropyl amine to the above solution;
c) heating the resulting solution upto 45 to 50° C.;
d) maintaining the reaction mass for about 55 to 100 hours;
e) cooling the above solution up to 25 to 35° C.;
f) separated solids were filtered and washed with methanol;
g) slurry wet filtered solids with water for about 30 to 60 minutes at 25 to 35° C.;
h) filtered the solids, washed with water and then spin dried for 30 to 60 minutes;
i) wet compound further dried at 70 to 75° C. for 4 hours under reduced pressure;
j) charging the resulting compound in 5% aqueous ethanol;
k) heating the resulted mass up to 40 to 45° C. for complete dissolution;
l) maintain the above solution for about 20 minutes at the same temperature and filtered out under nitrogen atmosphere;
m) cooled the solution to 0 to 5° C. and then maintained for about 30 to 60 minutes;
n) solids were separated by centrifuging;
o) washing the separated solids with chilled 5% aqueous ethanol and spin dried for 30 to 60 minutes to get 3-[2-[4-(6-Fluoro-1,2-benzisoxazol-3-yl)-1-piperidinyl]-ethyl]-6,7,8,9-tetrahydro-2-methyl-4H-pyrido[1,2-a]pyrimidin-4-one.

2. The process according to claim 1 of step (a), wherein the solvent used is selected from C₁₋₄ straight or branched chain alcohols.

3. The process according to claim 2, wherein the alcohol used is methanol.

4. The process according to claim 1 of step (b), wherein the organic base used is tertiary amine base.

5. The process according to claim 4, wherein the tertiary amine base used is diisopropyl ethylamine.

6. The process according to claim 1 of step (c), wherein the temperature ranging from 45-50° C.

7. The process according to claim 1 of step (j), wherein the ethanol containing 5% of water.

8. The process according to claim 1 of step (o), wherein the product was washed with chilled 5% aqueous ethanol.

9. The process according to claim 1, where in the final product of the present inventive substance has purity 99.6% or above.

10. The process according to claim 1, where in the final product of the present inventive substance has single maximum impurity less than 0.1%.

11. The process according to claim 1, where in the final product of the present inventive substance has 3-[2-[4-(6-Fluoro-1,2-benzisoxazol-3-yl)-1-piperidinyl]-ethyl]-9-hydroxy-6,7,8,9-tetrahydro-2-methyl-4H-pyrido[1,2-a]pyrimidin-4-one (9-hydrxy impurity) about 0.0025% or less.

12. The product obtained as per the claim 1 is free from the residual solvent methanol.

13. A process according to claim 1, wherein the final product of the present inventive substance is having moisture about 0.2% or less.

14. A pharmaceutical composition contains 3-[2-[4-(6-Fluoro-1,2-benzisoxazol-3-yl)-1-piperidinyl]ethyl]-6,7,8,9-tetrahydro-2-methyl-4H-pyrido[1,2-a]pyrimidin-4-one, according to claim 1 as active ingredient.
